(19) United States
(12) Patent Application Publication
(10) Pub. No.: US 2026/0174864 A1
(43) Pub. Date: Jun. 25, 2026

(54) TRANSDERMAL COMPOSITIONS AND METHODS OF PREPARING SAID COMPOSITIONS

(71) Applicant: Davies Pharmaceuticals LLC, Crown Point, IN (US)

(72) Inventor: Robb Davies, Crown Point, IN (US)

(73) Assignee: Davies Pharmaceuticals LLC, Crown Point, IN (US)

(21) Appl. No.: 19/541,600

(22) Filed: Feb. 17, 2026

Related U.S. Application Data
(63) Continuation-in-part of application No. 18/479,245, filed on Oct. 2, 2023.
(60) Provisional application No. 63/379,049, filed on Oct. 11, 2022.

(51) Int. Cl. A61K 45/06; A61K 8/04; A61K 9/00; A61K 9/70

(52) U.S. Cl. CPC A61K 45/06; A61K 8/042; A61K 9/0014; A61K 9/0056; A61K 9/7061

(57) ABSTRACT

Disclosed herein are transdermal compositions that are not transdermal patches, processes for preparing such transdermal compositions, and solvent/carrier compositions for such transdermal compositions.

### CROSS-REFERENCE TO RELATED APPLICATIONS

This application is a continuation-in-part (CIP) and claims the benefit of application Ser. No. 18/479,245, filed Oct. 2, 2023, which claims the benefit of U.S. Provisional Application No. 63/379,049, filed Oct. 11, 2022, the contents of which are incorporated by reference herein in their entirety.

### BACKGROUND OF THE INVENTION

Disclosed herein are transdermal compositions that are not transdermal patches, processes for preparing such transdermal compositions, and solvent/carrier compositions for transdermal compositions. Many diseases can be treated using the solvent/carrier compositions described herein. These solvent/carrier compositions employ dispersion bonding to link the solvent/carrier to the medication, which allows easy diffusion of the medication past the layers of the dermis, as a method to deliver each medication to the body and treat each unique disease.

The bonds that are created inside the body with each medication are much stronger as dispersion bonding is the weakest form of chemical bonding.

In 1996, Donepezil was approved for the treatment of dementia in a wide range of Alzheimer's patients, ranging from mild to severe cases. Donepezil has been prescribed and administered to Alzheimer's patients orally, which presents a number of problems. For example, adverse side effects resulting from oral doses of donepezil commonly include gastrointestinal issues, such as nausea, diarrhea, and vomiting.

Further, oral medications can be particularly problematic for patients suffering from for example, Alzheimer's disease. Due to the loss of cognition and/or behavioral changes that afflict many Alzheimer's patients, patients commonly refuse oral medication or spit out medications after they are administered. In an effort to alleviate these issues, transdermal therapies have been investigated. Most recently, a seven-day donepezil transdermal patch was approved by the FDA for the treatment of Alzheimer's Disease.

However, donepezil transdermal patches have their own host of problems associated with treatment. Specifically, transdermal patches have been shown to induce skin irritation in the area of application. These application-site reactions can cause itching, redness, and/or irritation. Additionally, as transdermal patches require constant adhesion to the patient's skin for continual dosing, application-site reactions can cause the patient to itch and scratch at the patch. If the patch is removed by the patient because it itches, then the patient is no longer receiving beneficial treatment.

Thus, there is a need for improved transdermal therapies using active ingredients (for example, donepezil) as well as improved methods for the preparation of such therapies. The present disclosure provides such improved therapies and methods.

US2021/0212923A1 relates to a cosmetic composition for topical application to the skin comprising, in a physiologically acceptable medium, at least an effective amount of at least one aqueous extract of rose and of at least one oily extract of rose, and the use thereof in particular for promoting the natural rhythmic process of skin cells and/or improving the micro-nutritional balance of the skin.

US2023/0364175A1 discloses an herbal composition including Lonicera Japonica and Andrographis Paniculata, a method of preparing the herbal composition, and a method of administering the herbal composition to a subject in need thereof is disclosed. The method of administering can include intranasal administration.

US2024/0108673A1 discloses a method for treating skin. The method can include topically applying to skin in need thereof a composition comprising effective amounts of Arctium lappa root extract and Epilobium angustifolium extract to reduce expression of a pro-inflammatory cytokine of IL-8, IL-2, or TNF-a in the skin.

JP2002322044A discloses a bleaching agent and composition for bleaching that include at least one kind of a dried product or an extract of a fruit body of a basidiomycete selected from the genera Flammulina and Hypsizigus of the family Tricholamataceae. The basidiomycete may be a Flammulina velutipes or Hypsizigus marmoreus. These bleaching agent and composition are excellent in safety and bleaching effect.

JP2003081840A discloses a refreshing preparation to impart the skin or mucosa or the like with refreshing feeling without being accompanied by unpleasant smell and/or local irritancy.

### BRIEF DESCRIPTION OF THE DRAWINGS

FIG. 1 1 depicts a cross-sectional view of human skin with an embodiment of the present disclosure applied thereto.

FIG. 2 is a scheme illustrating an embodiment of a process for preparing a transdermal composition comprising rose water, avocado oil, beeswax, glycerin, and donepezil.

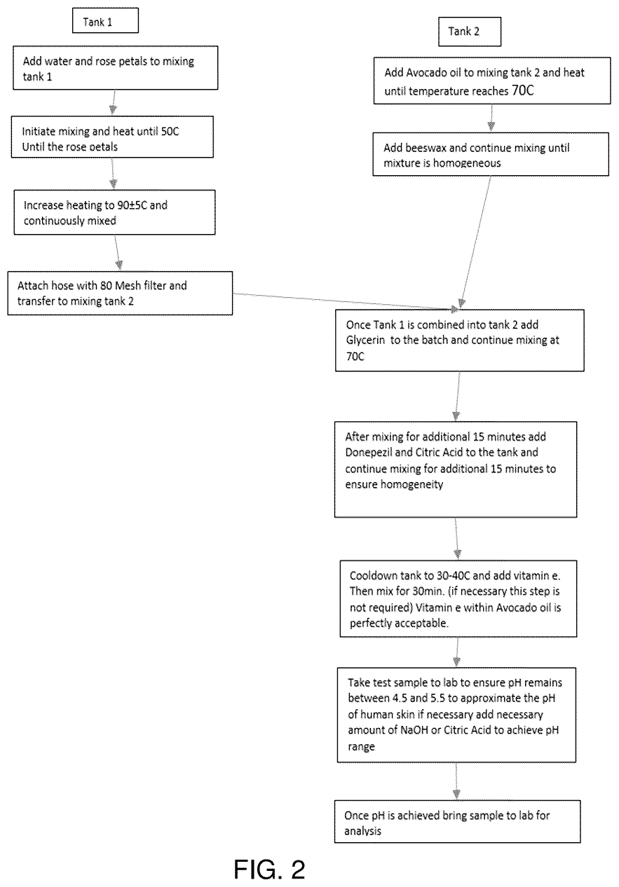

FIG. 3 is a scheme illustrating an embodiment of a process for preparing a transdermal composition comprising petroleum gel, glycerin, and donepezil.

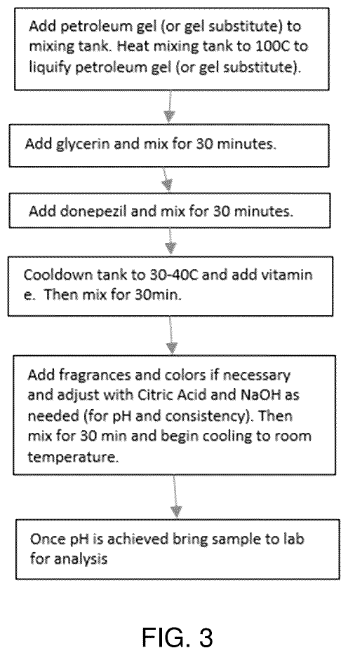

### DETAILED DESCRIPTION

### Definitions

As used herein, “a” or “an” entity refers to one or more of that entity, e.g., “a compound” refers to one or more compounds or at least one compound unless stated otherwise. As such, the terms “a” (or “an”), “one or more,” and “at least one” are used interchangeably herein.

As used herein, the term “about” means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%.

As used herein, a “pharmaceutically acceptable derivative” refers to a compound that is structurally modified from a parent drug but remains safe, non-toxic, and effective for use in humans or animals. These derivatives are typically designed to enhance the pharmacological properties of the original compound, such as improving solubility, bioavailability, stability, or reducing adverse effects. Examples of such derivatives include pharmaceutically acceptable salts, esters and amides of carboxylic acid functional groups within the parent molecule or variations of the parent molecule that possess similar or improved therapeutic properties of the parent compound.

As used herein, “a therapeutically effective amount” of a compound disclosed herein refers to an amount of the compound that will elicit a biological or medical response in a subject. The therapeutically effective amount will depend on the purpose of the treatment and will be ascertainable by one of ordinary skill in the art (see, e.g., Lloyd (1999) The Art, Science and Technology of Pharmaceutical Compounding).

As used herein, the term “inhibit,” “inhibition,” or “inhibiting” refers to the reduction or suppression of a given condition, symptom, or disorder, or disease, or a significant decrease in the baseline activity of a biological activity or process.

As used herein, the term “treat,” “treating,” or “treatment,” when used in connection with a disorder or condition, includes any effect, e.g., lessening, reducing, modulating, ameliorating, or eliminating, that results in the improvement of the disorder or condition. Improvements in or lessening the severity of any symptom of the disorder or condition can be readily assessed according to standard methods and techniques known in the art.

As used herein, a “mammal” refers to domesticated animals (e.g., dogs, cats, and horses) and humans. In some embodiments, the mammal is a human. In some embodiments, the mammal is a canine.

For the purposes of promoting and understanding the principles of the invention, reference will now be made to the embodiment illustrated in the drawings and specific language will be used to describe the same. It will nevertheless be understood that no limitation of the scope of the invention is thereby intended. Any alterations and further modifications in the described embodiments, and any further applications of the principles of the invention described herein are contemplated herein as would be apparent to one of ordinary skill in the art.

Gels, creams, and gel-creams of the present disclosure include a solvent phase or carrier used to deliver the example active ingredient, donepezil. The solvent, also called solvent phase or carrier, may be comprised of a number of different types of compounds. For example, the solvent may include humectants, moisturizing agents, UV light blockers, skin and muscle fortifying agents, nourishing agents, emulsifiers, sources of antioxidants, fragrances, and/or water, among others. The solvent phase is formulated to properly deliver the example API, donepezil, while reducing adverse side effects of other delivery methods, such as irritated skin at the application site.

The solvent of the transdermal therapy of the present disclosure may include one or more humectants. Humectants absorb and maintain moisture from the surrounding air. Humectants come in a variety of types. For example, humectants may be natural, naturally derived, or synthetically made. Specifically, humectants which may be used include beeswax, aloe vera, hyaluronic acid, petroleum gel, honey, and glycols, among others. The solvent phase or the “solvent/carrier composition” as used herein may include one or more of these humectants and combinations thereof. Alternatively, “petroleum gel” or “beeswax” in any of the disclosed processes or transdermal compositions described herein may be replaced with any of the humectants. In one embodiment, the humectant comprises any of the humectants mentioned herein with the addition of (i.e., along with) dimethyl sulfoxide (DMSO).

The solvent may include skin and muscle fortifying/anti-inflammatory agents. Beeswax and Vitamin E are also strong fortifying/anti-inflammatory agents for skin and muscles.

In addition, the solvent of the transdermal therapies of the present invention may include moisturizing agents included to assist in the repair of damaged or dry skin. For example, the solvent may include glycerin. The glycerin has the added benefit of maintaining the aroma of any fragrance added to the solvent and acting as an emulsifying agent. Alternatively, or additionally, the solvent may include petroleum gel. The solvent may also include antioxidants and/or a source of antioxidants. For example, the solvent may include Vitamin A, Vitamin C, Vitamin E, and/or other antioxidants. In other embodiments, the solvent may include sources of antioxidants, such as vegetable oils, e.g., avocado oil.

In addition, the formulation may have skin repairing, nourishing, and protecting agents. Roses, beeswax, and aloe vera also help to repair irritated or damaged skin. Vitamin E is used to nourish and protect skin cells from damage.

There may also be ingredients to protect skin specifically against UV light. For example, Beeswax may provide mild to intense UV light protection.

The solvent may further include a source of fragrance. In some embodiments, the fragrance may be a pre-formulated product/synthetic fragrance added during preparation. In other embodiments, the fragrance may be a naturally occurring or naturally derived fragrance. For example, essential oils, extracts, or components of flowers, such as rose petals, may be used to impart a pleasant fragrance.

The solvent may also include a colorant. The colorants may be used to designate the therapeutic strength of the transdermal therapy of the present disclosure. For example, a colorless/white cream or gel may indicate that application of a particular size (e.g. 2-3 ml) aliquot will deliver a 5 mg dose of donepezil, while a light blue cream may indicate delivery of a 10 mg dose of the exemplified API, donepezil, while a light blue cream or gel may indicate delivery of a 10 mg dose of donepezil when using the same size aliquot (2-3 ml). Such color-coded creams/gels will help ensure that patients, caregivers, and/or healthcare professionals are applying the proper amount of cream/gel that is required for the patient to receive a particular dosage.

Other ingredients may also be utilized in the creams and gels of the present disclosure. For example, water and/or rose-water may be used.

As will be understood by one of ordinary skill in the art to which the present disclosure relates, various quantities, ratios, and or techniques may be utilized in the practice of the present disclosure.

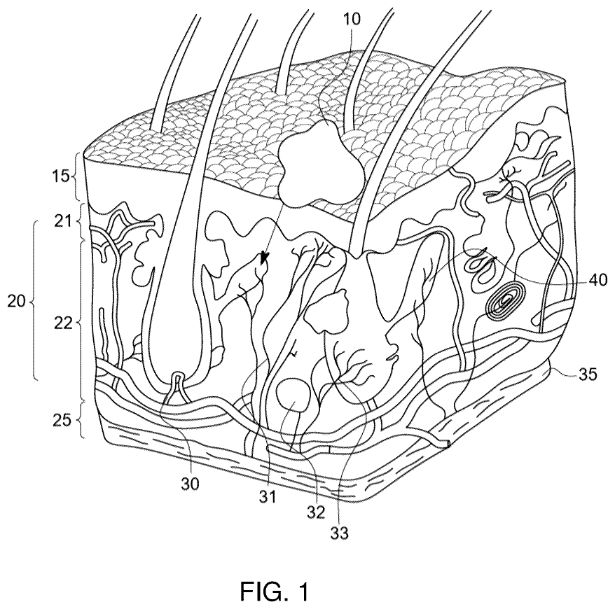

FIG. 1 depicts a cross-sectional view of human skin with an aliquot 10 of an embodiment of the present disclosure applied thereto. The human skin consists of three layers: the epidermis 15, the dermis 20, and the hypodermis 25. The dermis 20 can be further subdivided into the papillary layer 21 and the reticular layer 22. Within the layers of the human skin are nervous system structures 30 and muscles, for example, the arrector pili muscle 40. The nervous system structure 30 includes sensory nerve fibers 31, Pacinian corpuscle 32, and hair follicle receptors 33. Below the three layers of human skin lies the skeletal muscle 35.

As shown in FIG. 1, aliquot 10 of an embodiment of the present disclosure is applied to the epidermis 15. The aliquot 10 is absorbed into the skin, providing any moisturizing, nourishing, protective, and or fragrance properties contained in the aliquot 10. As such, the application site will not become irritated, etc. Further, the exemplified API, donepezil (not shown separately) is delivered to the skeletal muscular junction where it prevents acetylcholinesterase from breaking down acetylcholine. The additional acetylcholine present then allows for better communication between nerve cells. The excess breakdown of acetylcholine is thought to cause the dementia and or behavioral symptoms associated with Alzheimer's disease.

In order to promote a further understanding of the present invention and its various embodiment, the following specific examples are provided. It will be understood that these examples are illustrative and are not meant in any way to limit the scope of the disclosure.

### Example 1: Preparation of Donepezil Transdermal Therapy Rose Gel-Cream

### Materials and Methods

The preparation of this exemplary cream utilizes two vessels or tanks. The two vessels or tanks may be prepared simultaneously or consecutively. However, the contents of Tank 1 and Tank 2 will be added together. For the preparation of Tank 1, water and rose petals are added to a clean tank. The rose petals and water are mixed, and the contents are heated for thirty minutes at 50 degrees C. Once the rose petals are wilted, the tank is then heated to approximately 90 plus or minus 5 C, with continuous mixing. This creates rose water.

For the preparation of Tank 2, avocado oil is added to the Tank and mixed. The tank is then heated to 70 degrees C. Once the Tank reaches temperature, beeswax is added with continuous mixing. The mixing will continue until the contents of Tank 2 are homogeneous.

Once Tank 1 and Tank 2 are properly prepared, and the contents of each are mixed together. This is accomplished by filtering the contents of Tank 1 through an 80-mesh sieve and pumping the filtered Tank 1 material into Tank 2. Once the contents of Tank 1 are transferred to Tank 2. Glycerin is then added to Tank 2 with continued mixing and heating to 70 degrees C., to be used as an emulsifier to homogenize the mixture of components within Tank 2. After fifteen minutes, the correct dosage of donepezil is added to the mixture. The contents in Tank 2 are mixed for an additional fifteen minutes to ensure homogeneity. After completion of this mixing step, the contents are continuously mixed and cooled to room temperature until the solution achieves a semi-sold state. Colorants and/or fragrance may be added to the gel-cream with additional mixing.

If necessary, the pH of the resulting gel-cream can be adjusted. In one preferred embodiment, the pH of the gel-cream is around 4.5 to 5.5, the approximate pH of human skin. Adjustment of the pH of the solution can be accomplished using either sodium hydroxide (NaOH) or citric acid, as needed. Citric acid or sodium hydroxide may also be added after the addition of donepezil to maintain consistency of the product.

The amounts of each compound or ingredient in the compound will vary depending on the desired dosage of the exemplified API, donepezil that is to be included in the resulting gel-cream. Additionally, the heating and mixing may be accomplished by any suitable means known to the hose skilled in the art in this field (e.g., a heating plate/mixing plate for small batches or industrial sized heaters and mixers for large batches).

### Example 2: Preparation of Donepezil Transdermal Therapy Gel

### Materials and Methods

Petroleum gel is added to a mixing tank and heated to 100 C to ensure the petroleum gel liquefies. After 30 minutes of mixing, Glycerin is added to the mixing tank. The tank is mixed for an additional 30 minutes, then the appropriate amount of the exemplified API, donepezil is added to achieve the target concentration of the batch. The tank is then cooled to 35 C and Vitamin E extract is added. The solution is then mixed for at least 30 minutes. If desired, fragrance and/or colorants can be added to the solution at this point. Then, the solution is continuously mixed and cooled to room temperature until a gel consistency is achieved.

If necessary, the pH of the resulting gel can be adjusted. In one embodiment, the pH of the gel is around 4.5 to 5.5, the approximate pH of human skin. Adjustment of the pH of the solution can be accomplished using either Sodium Hydroxide (NaOH) or Citric Acid, as needed. Citric acid or Sodium Hydroxide may also be added after the addition of the exemplified API, donepezil to maintain consistency of the product.

The amounts of each compound or ingredient utilized in the preparation of Example 2 will vary depending on the strength/dosage of the exemplified API, donepezil that is desired for the batch.

### Example 3: Method of Delivery Utilizing Example 1 or Example 2

### Materials and Methods

The transdermal therapy compounds from Example 1 or Example 2 are used for the treatment of an exemplified disease, Alzheimer's Disease. A patient presents with both cognitive deterioration and behavioral issues as a result of Alzheimer's Disease. A 2-3 mL aliquot of the transdermal therapy is applied to the patient's skin, preferably on their arm, thigh, buttocks, or back, which overlays skeletal muscle, daily using a formulation of the required strength (i.e., 10 mg formulation for a 10 mg prescription). Within 2-3 days after daily application of the transdermal therapy, cognitive and behavioral symptoms are lessened. Within 3-5 weeks of daily application of the transdermal therapy, patient's symptoms are lessened further. Example 1 will add UV light protection. Examples 1 and 2 will also reduce/eliminate any skin irritation or itching, burns, joint pain, or wounds by the inclusion of the moisturizing agents, nourishing agents, UV light protection agents, fortifying agents, skin protecting agents, humectants, and/or antioxidants in the transdermal therapy.

### Example 4: Method of Delivery of Donepezil Utilizing Example 1 or Example 2

### Materials and Methods

The transdermal therapy compounds from Example 1 or example 2 are used for the treatment of the exemplified disease, Alzheimer's Disease. A patient presents with both cognitive deterioration and behavioral issues as a result of Alzheimer's disease. A 2-3 mL aliquot of the transdermal therapy is applied to the patient's skin, preferably their arm, thigh, buttocks, or back, which overlays skeletal muscle, twice daily using a formulation of half of the required strength (i.e. 5 mg formulation for a 10 mg prescription). The transdermal therapy is applied both in the morning and at night. Within 2-3 days after twice daily application of the transdermal therapy, cognitive and behavioral symptoms are lessened. Within 3-5 weeks of twice daily application of the transdermal therapy, patient's symptoms are lessened further. Example 1 will add UV light protection. Example 1 and 2 will also reduce/eliminate any skin irritation, itching, or joint pain by the inclusion of the moisturizing agents, nourishing agents, fortifying agents, skin protecting agents, humectants, and or antioxidants present in the transdermal therapy.

Note that the aliquot size used to administer an appropriate dose is dependent upon the concentration (i.e. mg/mL) of donepezil in the transdermal therapy and how it is prepared. The aliquot sizes in Examples 3 and 4 are illustrative.

Additional Embodiments of the disclosure are set forth below.

### Additional Embodiments

Non-limiting embodiments of this disclosure include:
Embodiment 1: A process for preparing a transdermal composition comprising the steps of: a) boiling flower petals in water to prepare flower water at about 85° C. to about 95° C. in Container 1; b) heating a vegetable oil with beeswax or beeswax/dimethyl sulfoxide (DMSO) mixture at about 65° C. to about 75° C. in Container 2; c) adding the contents of Container 1 to Container 2 by filtering the contents of Container 1 through a sieve into Container 2 and mixing the resultant mixture; d) thereafter, adding glycerin to Container 2 with continued mixing and heating to a temperature of about 65° C. to about 75° C.; e) thereafter, adding an active pharmaceutical ingredient (API) to the mixture of step d); and f) cooling the mixture of step e) to room temperature such that a semi-solid final mixture is obtained; wherein the transdermal composition is not in the form a transdermal patch.
Embodiment 2: The process of Embodiment 1, further comprising cooling the mixture of step d) to a temperature from about 30° C. to about 40° C., and adding Vitamin E prior to proceeding with step e).
Embodiment 3: The process of Embodiment 1, wherein for the preparation of about 100 g of the transdermal composition: In step a), from about 0.5 grams to about 1.5 grams of flower petals, and from about 16.9 grams to about 17.9 grams of water are used; In step b) from about 60.9 grams to about 61.9 grams of vegetable oil, and from about 13.6 grams to about 14.6 grams of beeswax or beeswax/DMSO mixture are used; and the beeswax/DMSO mixture if used contains from about 6.80 grams to about 13.432 grams of beeswax and from about 1.088 grams to about 7.3 grams of DMSO; In step d), from about 5.64 grams to about 6.64 grams of glycerin is used; and In step e), from about 0.000500 grams to about 0.200 grams of the API is used.
Embodiment 4: The process of Embodiment 2, wherein after cooling the mixture of step d) to a temperature from about 30° C. to about 40° C., from about 0.007 g to about 0.4 g of Vitamin E is added prior to proceeding with step e). In one embodiment, the amount of Vitamin E added is at a minimum the amount of Vitamin E that is present intrinsically in avocado oil, and at a maximum the amount of Vitamin E that is present intrinsically in wheat germ oil.
Embodiment 5: A transdermal composition comprising: a) from about 0.0005% to about 0.20% by weight of at least one active pharmaceutical ingredient (API); b) from about 17.9% to about 18.9% by weight of an aqueous flower petal solution or mixture, wherein the aqueous flower petal solution or mixture comprises from about 0.500% to about 1.50% by weight of flower petals and from about 16.9% to about 17.9% by weight of water; c) from about 6.9% to about 61.9% by weight of a vegetable oil; d) from about 13.6% to about 14.6% by weight of beeswax or beeswax/DMSO mixture, wherein the beeswax/DMSO mixture if used, comprises from about 50.0% to about 92.0% by weight of beeswax and from about 8.0% to about 50.0% by weight of DMSO; and e) from about 5.64% to about 6.64% by weight of glycerin, wherein the transdermal composition is not in the form of a transdermal patch.
Embodiment 6: The transdermal composition of Embodiment 5, further comprising: f) from about 0.007% to about 0.400% by weight of Vitamin E.
Embodiment 7: A solvent/carrier composition for a transdermal composition comprising: a) from about 17.9% to about 18.9% by weight of an aqueous flower petal solution or mixture, wherein the aqueous flower petal solution or mixture comprises from about 0.5% to about 1.5% by weight of flower petals and from about 16.9% to about 17.9% by weight of water; b) from about 60.9% to about 61.9% by weight of a vegetable oil; c) from about 13.6% to about 14.6% by weight of beeswax or beeswax/DMSO mixture, wherein the beeswax/DMSO mixture if used, comprises from about 50% to about 92% by weight of beeswax and from about 8% to about 50% by weight of DMSO; and d) from about 5.64% to about 6.64% by weight of glycerin; wherein the transdermal composition is not in the form of a transdermal patch.
Embodiment 8: The solvent/carrier composition of Embodiment 8, further comprising: e) from about 0.007% by to about 0.400% by weight of Vitamin E.
Embodiment 9: A process for preparing a transdermal composition comprising the steps of: a) heating petroleum gel or petroleum gel/DMSO mixture to a temperature of about 95° C. to about 105° C. to liquify the petroleum gel; b) thereafter, adding glycerin c) thereafter, adding an active pharmaceutical ingredient (API) to the mixture of step b); d) cooling the mixture of step c) to about 30° C. to 40° C.; e) adding Vitamin E to the mixture of step d); and f) further cooling the mixture of step e) to room temperature such that a semi-solid final mixture is obtained.
Embodiment 10: The process of Embodiment 9, wherein for the preparation of about 100 g of the transdermal composition: In step a), from about 74.0 grams to about 84.0 grams of petroleum gel, and if petroleum gel/DMSO mixture is used, then the amount of petroleum gel in the petroleum gel/DMSO mixture is about 37.0 grams to about 67.2 grams and the amount of DMSO in the mixture is about 14.8 grams to about 42.0 grams; In step b), from about 6.0 grams to about 7.0 grams of glycerin; In step c), from about 0.00050 grams to about 0.20 grams of the API is used; and In step e), from about 10.0 grams to about 20.0 grams of Vitamin E is used.
Embodiment 11: A transdermal composition comprising: a) from about 0.0005% to about 0.20% by weight of an active pharmaceutical ingredient (API); b) from about 74% to about 84% by weight of petroleum gel or petroleum gel/DMSO mixture; c) from about 6% to about 7% by weight of glycerin; and d) from about 10% to about 20% by weight of Vitamin E; wherein the transdermal composition is not in the form of a transdermal patch.
Embodiment 12: The process of any of Embodiments 1-8, wherein the flower petals are from a fragrant or nonfragrant flower, i.e., the flower petals can be obtained from any flower.
Embodiment 13: The process of Embodiment 12, wherein the fragrant flower is at least one chosen from rose, jasmine, Gardenia, lavender, lilac, honeysuckle, lily of the valley, hyacinth, peony, freesia, Berlandiera, Agave, Dianthus, Nicotiana, Hedychium, tuberose, sweat pea, sweet alyssum, Gardenia jasminoides (commonly known as gardenia and cape jasmine), phlox, moonflower, stephanotis, and dandelions. These are some nonlimiting examples of the fragrant flower. In one preferred embodiment, the fragrant flower is rose.
Embodiment 14: The process of Embodiment 12, wherein the nonfragrant flower is at least one chosen from anemones, birds of paradise, bougainvillea, calla lilies, chrysanthemums, dahlias, echinacea, gladiolus, hibiscus, hydrangeas, listanthus, orchids, poppies, ranunculus, scarlet gilia, sunflowers, and tulips. These are some nonlimiting examples of the non-fragrant flower.
Embodiment 15: The process of any of Embodiments 1-6, wherein the vegetable oil is at least one chosen from avocado oil, wheat germ oil, sunflower oil, safflower oil, almond oil, hazelnut oil, peanut oil, soybean oil and corn oil. These are nonlimiting some non-limiting examples of the vegetable oil. In one preferred embodiment, the vegetable oil is avocado oil. It is worthwhile mentioning that all of these vegetable oils intrinsically contain various amounts of Vitamin E in them. As an example, avocado oil, intrinsically contains from about 0.007% to about 0.033% by weight of Vitamin E. Similarly, the vitamin E content in some of the other vegetable oils are as follows: Wheat Germ Oil: ˜0.1% to ˜0.3%; Sunflower Oil: ˜0.041% to 0.0938%; Safflower Oil: 0.034% to 0.103%; Almond Oil: ˜0.039% to 0.088%; Cottonseed Oil: ˜0.035% to 0.106%; Grapeseed Oil: ˜0.001% to 0.053%; Canola Oil: ˜0.017% to 0.07%; Olive Oil: ˜0.01% to 0.062; Corn Oil: ˜0.082% to 0.183%; and Soybean Oil: ˜0.03%. to 0.16%.
Embodiment 16: The process of any of Embodiments 1-6, and 9-11, wherein the API is at least one chosen from donepezil, galanthamine, rivastigmine, tacrine, aripiprazole, clozapine, haloperidol, olanzapine, quetiapine, risperidone, ziprasidone, fluphenazine, prochlorperazine, thioridazine, thiothixene, aspirin, melphalan, chlorambucil, cyclophosphamide, busulfan, thiotepa, dacarbazine, methotrexate, hydroxyurea, formononetin, calycosin, carbamazepine, lorazepam, clonidine, cyclobenzaprine, dexamethasone, gabapentin, lamotrigine, levetiracetam, phenytoin, riluzole, topiramate, valproic acid, temozolomide, cyclophosphamide, lomustine, cisplatin, oxaliplatin, carboplatin, 5-Fluorouracil (5-FU), tamoxifen, adderall, lithium, and a pharmaceutically acceptable derivative thereof. These are some non-limiting examples of the API. In one preferred embodiment, the API is at least one chosen from donepezil, galanthamine, rivastigmine, tacrine, or a pharmaceutically acceptable derivative thereof. In another preferred embodiment, the API is donepezil or a pharmaceutically acceptable derivative thereof.

The amount of API used in the processes and transdermal compositions of the disclosure varies and depends on the identity and different dosages of medication. In one embodiment, in the processes of this disclosure, assuming the production of 100 grams of the transdermal composition, the amount of API will vary from 0.0005 grams (0.5 mg prescription) to 0.20 grams (200 mg prescription), and in one embodiment, 0.001 grams (1 mg prescription), 0.005 g (5 mg prescription), 0.010 grams (10 mg prescription), 0.020 grams (20 mg prescription), 0.030 grams (30 mg prescription), 0.050 grams (50 mg prescription), 0.060 grams (60 mg prescription), 0.100 grams (100 mg prescription), and 0.200 grams (200 mg prescription).
Embodiment 17: The process of embodiment 1 or 2, wherein in step c), the sieve is at least one chosen from 80 mesh, 70 or lower mesh, and 100 mesh sieve. In a preferred embodiment, the sieve is an 80 mesh sieve.
Embodiment 18: The process of any of embodiments 1-4, and 9-10, further comprising the step of adding to the mixture of the final step one or more colorants and/or one or more fragrances.
Embodiment 19: The process of any of embodiments 1-4, and 9-10, further comprising adjusting the pH of the mixture after the final step to a value from about 4.2 to about 5.8 by the addition of an acid or base. In one embodiment, the pH of the mixture after the final step is adjusted to a value from about 4.5 to about 5.5.
Embodiment 20: The process of Embodiment 19, wherein the acid is a weak organic acid.
Embodiment 21: The process of Embodiment 20, wherein the weak organic acid is a polyfunctional organic acid or a metal salt thereof.
Embodiment 22: The process of Embodiment 21, wherein the polyfunctional organic acid or the metal salt thereof is at least one chosen from citric acid, carbonic acid, succinic acid, malic acid, tartaric acid, 1,2,3,4-butanetetracarboxylic acid, tannic acid, glutaric acid, ascorbic acid (vitamin C), lactic acid, acetic acid (vinegar), tartaric acid, and a metal salt of any of these organic acids.
Embodiment 23: The process of Embodiment 22, wherein the polyfunctional organic acid metal salt is at least one chosen from sodium or potassium glutamate, sodium or potassium tannate, sodium or potassium citrate, sodium or potassium acetate, sodium or potassium lactate, sodium or potassium malate, and sodium or potassium tartrate.
Embodiment 24: The process of Embodiment 19, wherein the acid is an inorganic acid, and is chosen from at least one of hydrochloric acid, sulfuric acid, boric acid, and phosphoric acid.
Embodiment 25: The process of Embodiment 19, wherein the base is an inorganic or organic base.
Embodiment 26: The process of Embodiment 25, wherein the inorganic base is chosen from at least one of sodium, potassium, calcium, magnesium or ammonium hydroxide; sodium, potassium, calcium, magnesium, or ammonium bicarbonate; sodium, potassium, calcium, magnesium or ammonium phosphate; aluminum silicate; and silica.
Embodiment 27: The process of Embodiment 3 or 10, wherein about 0.005 grams or about 0.010 grams of API is used.
Embodiment 28: The transdermal composition of Embodiment 5 or 10, wherein the API comprises about 0.005% to about 0.010% by weight of the transdermal composition.
Embodiment 29: The process of any of Embodiments 1-4, and 9-10, wherein the semi-solid final mixture is in the form of a gel, a cream, or a gel-cream.
Embodiment 30: A transdermal composition made by the process of any one of Embodiments 1-4, and 9-10.
Embodiment 31: A method of treating a disease, comprising administering to the subject in need of such treatment an effective amount of the transdermal composition of any of Embodiments 5-6, 11, and 30.
Embodiment 32: The method of Embodiment 31, wherein the disease is at least one chosen from mania, schizophrenia, bipolar disorder, autism, major depressive disorder, Tourette syndrome, hallucinations, delusions, disorganized thinking, schizoaffective disorder, suicidal thoughts, Parkinson's disease, treatment resistant mania, treatment resistant schizophrenia, psychosis in Parkinson's disease, catatonia, treatment resistant catatonia, severe behavior problems, severe hyperexcitability, severe hyperactivity, acute agitation, acute anxiety, psychosis, delirium, nausea, vomiting, nausea and vomiting caused by chemotherapy, acute Mania, bipolar I disorder, manic or mixed episodes, depressive episodes, mood maintenance in bipolar I disorder, treatment resistant depression, cancer, cachexia, acute depressive episodes in adults with bipolar I or II disorder, bipolar maintenance to avoid return of symptoms, major depressive disorder, generalized anxiety disorder, insomnia, post-traumatic stress disorder(PTSD), borderline personality disorder(BPD), obsessive compulsive disorder (OCD), irritability associated with autistic disorder, severe aggression or disruptive behavior disorders not associated with autism, disorganized thinking or speech, agitation, hostility, severe nervousness, social withdrawal or lack of motivation, Huntington's disease, migraine headaches, vertigo, balance problems, motion sickness, long and short term depression or anxiety disorders, pediatric behavior disorders, anxiety, tension, sleep disturbances, dementia, dementia related psychosis, long term movement disorders, tardive dyskinesia, neuroleptic malignant syndrome, mild to moderate pain relief, fever reduction, inflammation, arthritis, juvenile idiopathic arthritis, pericarditis, Kawasaki disease, cardiac attack, stroke, angina, preeclampsia, multiple myeloma, ovarian cancer, uveal melanoma, malignant lymphoma, myeloblastic leukemia, childhood neuroblastoma, breast cancer, amyloidosis, localized malignant melanoma, soft tissue sarcoma, chronic lymphocytic leukemia (CLL), Hodgkin lymphoma, non-Hodgkin lymphoma(NHL), Waldenström's macroglobulinemia, kidney disease, mycosis fungoides, cutaneous T-cell lymphoma, leukemia, acute lymphoblastic leukemia (ALL), acute myeloid leukemia (AML), and chronic myelogenous leukemia (CML), retinoblastoma sarcoma, Ewing's sarcoma, small cell lung cancer, non-small cell lung cancer, nephrotic syndrome in children, systemic lupus erythematosus (SLE), lupus nephritis, vasculitis, systemic sclerosis (scleroderma), myopathy, dermatomyositis, rheumatoid arthritis, multiple sclerosis, polycythemia vera, essential thrombocythemia, myelofibrosis, bladder cancer, malignant effusions, metastatic malignant melanoma, osteogenic sarcoma, malignant glucagonoma, rheumatoid arthritis, psoriasis, psoriatic arthritis, polyarticular juvenile idiopathic arthritis (PJJA), lupus, multiple sclerosis, vasculitis, lung cancer, head and neck cancer, osteosarcoma, mycosis fungoides, gestational trophoblastic tumors, traumatic brain injury (TBI), spinal cord injury (SCI), ischemic stroke, allergic inflammation, autoimmune hepatitis, pulmonary hypertension, osteoporosis, epilepsy, trigeminal neuralgia, attention deficit hyperactivity disorder (ADHD), hypertension, pain, severe pain associated with cancer, opioid and alcohol withdrawal syndrome, menopausal hot flashes, Tourette's syndrome, migraine, strains, sprains, acute back pain, acute neck pain, fibromyalgia, myofascial pain, dermatitis, pemphigus, psoriasis, asthma, respiratory disorder, inflammatory bowel diseases, ulcerative colitis, regional enteritis, nervous system flare-ups, acute exacerbations of multiple sclerosis, cerebral edema, hypercalcemia, Cushing's disease, severe COVID-19, airway swelling, restless legs syndrome (RLS), nerve pain, diabetic neuropathy, hot flashes, chronic pain, trigeminal neuralgia, unipolar depression, partial-onset (focal) seizures, myoclonic seizures, primary generalized tonic-clonic seizures, status epilepticus, seizure, prevention, seizure in dogs and cats, ataxia, binge eating disorder, alcoholism, diabetic peripheral neuropathy, postherpetic neuralgia, glioblastoma multiforme (GBM), anaplastic astrocytoma (Grade 3 astrocytoma), neuroendocrine tumors, metastatic melanoma, pituitary carcinoma, adrenal gland cancer, brain cancer/tumor, testicular cancer, cervical cancer, esophageal cancer, stomach (gastric) cancer, malignant mesothelioma, colon cancer, colorectal cancer, pancreatic cancer, biliary tract (gallbladder and bile duct) cancer, endometrial cancer, Wilms' tumor, mesothelioma, anal carcinoma, actinic keratosis, and superficial basal cell carcinoma.

In one preferred embodiment, the disease is dementia.

In another embodiment, the dementia is at least one chosen from Alzheimer's disease, Creutzfeldt-Jakob disease, dementia with Lewy Bodies, Down syndrome, frontotemporal dementia, Huntington's disease, mixed dementia, normal pressure hydrocephalus, posterior cortical atrophy, Parkinson's disease dementia, vascular dementia, and Korsakoff syndrome.

In a preferred embodiment, the dementia is Alzheimer's disease.

In another preferred embodiment, the disease is cancer.

In another embodiment, the cancer is at least one chosen from ovarian cancer, breast cancer, lung cancer, non-small cell lung cancer, small cell lung cancer, bladder cancer, head and neck cancer, adrenal gland cancer, brain cancer/tumor, testicular cancer, stomach cancer, cervical cancer, esophageal cancer, colon cancer, colorectal cancer, pancreatic cancer, biliary tract cancer, endometrial cancer, and superficial basal cell carcinoma.

What is claimed is:

1. A process for preparing a transdermal composition comprising the steps of:
a) boiling flower petals in water to prepare flower water at about 85° C. to about 95° C. in Container 1;
b) heating a vegetable oil with beeswax or beeswax/dimethyl sulfoxide (DMSO) mixture at about 65° C. to about 75° C. in Container 2;
c) adding the contents of Container 1 to Container 2 by filtering the contents of Container 1 through a sieve into Container 2 and mixing the resultant mixture;
d) thereafter, adding glycerin to Container 2 with continued mixing and heating at about 65° C. to about 75° C.;
e) thereafter, adding an active pharmaceutical ingredient (API) to the mixture of step d); and
f) cooling the mixture of step e) to room temperature such that a semi-solid final mixture is obtained;
wherein the transdermal composition is not in the form a transdermal patch.

2. The process of claim 1, further comprising cooling the mixture of step d) to a temperature from about 30° C. to about 40° C., and adding Vitamin E prior to proceeding with step e).

3. The process of claim 1, wherein in step a), the flower petals are from a fragrant or nonfragrant flower.

4. The process of claim 3, wherein the fragrant flower is at least one chosen from rose, jasmine, gardenia, lavender, lilac, honeysuckle, lily of the valley, hyacinth, peony, freesia, Berlandiera, Agave, Dianthus, Nicotiana, Hedychium, tuberose, sweat pea, sweet alyssum, Gardenia jasminoides, phlox, moonflower, stephanotis, and dandelions.

5. The process of claim 3, wherein the fragrant flower is rose.

6. The process of claim 3, wherein the nonfragrant flower is at least one chosen from anemones, birds of paradise, bougainvillea, calla lilies, chysanthemums, dahlias, echinacea, gladiolus, hibiscus, hydrangeas, listanthus, orchids, poppies, ranunculus, scarlet gilia, sunflowers, and tulips.

7. The process of claim 1, wherein in step b), the vegetable oil is at least one chosen from avocado oil, wheat germ oil, sunflower oil, safflower oil, almond oil, hazelnut oil, peanut oil, soybean oil and corn oil.

8. The process of claim 6, wherein the vegetable oil is avocado oil.

9. The process of claim 1, wherein in step c), The contents of Container 1 is added to Container 2 by pumping the contents of Container 1 into Container 2.

10. The process of claim 1, wherein in step e), the API is at least one chosen from donepezil, galanthamine, rivastigmine, tacrine, aripiprazole, clozapine, haloperidol, olanzapine, quetiapine, risperidone, ziprasidone, fluphenazine, prochlorperazine, thioridazine, thiothixene, aspirin, melphalan, chlorambucil, cyclophosphamide, busulfan, thiotepa, dacarbazine, methotrexate, hydroxyurea, formononetin, calycosin, carbamazepine, lorazepam, clonidine, cyclobenzaprine, dexamethasone, gabapentin, lamotrigine, levetiracetam, phenytoin, riluzole, topiramate, valproic acid, temozolomide, cyclophosphamide, lomustine, cisplatin, oxaliplatin, carboplatin, 5-Fluorouracil (5-FU), tamoxifen, adderall, lithium, and a pharmaceutically acceptable derivative thereof.

11. The process of claim 10, wherein the API is at least one chosen from donepezil, galanthamine, rivastigmine, tacrine, or a pharmaceutically acceptable derivative thereof.

12. The process of claim 11, wherein the API is donepezil or a pharmaceutically acceptable derivative thereof.

13. The process of claim 1, wherein the sieve is at least one chosen from 80 mesh, 70 or lower mesh, and 100 mesh sieve.

14. The process of claim 1, further comprising the step of adding to the mixture of step f) one or more colorants and/or one or more fragrances.

15. The process of claim 1, further comprising adjusting the pH of mixture of step f) from about 4.2 to about 5.8 by the addition of an acid or base.

16. The process of claim 15, wherein the acid is a weak organic acid.

17. The process of claim 16, wherein the weak organic acid is a polyfunctional organic acid or a metal salt thereof.

18. The process of claim 17, wherein the polyfunctional organic acid or the metal salt thereof is at least one chosen from citric acid, carbonic acid, succinic acid, malic acid, tartaric acid, 1,2,3,4-butanetetracarboxylic acid, tannic acid, glutaric acid, ascorbic acid, lactic acid, acetic acid, tartaric acid, and a metal salt of any of these organic acids.

19. The process of claim 18, wherein the polyfunctional organic acid metal salt is at least one chosen from sodium or potassium glutamate, sodium or potassium tannate, sodium or potassium citrate, sodium or potassium acetate, sodium or potassium lactate, sodium or potassium malate, and sodium or potassium tartrate.

20. The process of claim 15, wherein the acid is an inorganic acid, and is chosen from at least one of hydrochloric acid, sulfuric acid, boric acid, and phosphoric acid.

21. The process of claim 15, wherein the base is an inorganic or organic base.

22. The process of claim 21, wherein the inorganic base is chosen from at least one of sodium, potassium, calcium, magnesium or ammonium hydroxide; sodium, potassium, calcium, magnesium, or ammonium bicarbonate; sodium, potassium, calcium, magnesium or ammonium phosphate; aluminum silicate; and silica.

23. The process of claim 1, wherein in step f), the final semi-solid mixture is in the form of a gel, a cream, or a gel-cream.

24. The process of claim 1, wherein for the preparation of about 100 g of the transdermal composition:
In step a), from about 0.5 grams to about 1.5 grams of flower petals, and from about 16.9 grams to about 17.9 grams of water are used;
In step b) from about 60.9 grams to about 61.9 grams of vegetable oil, and from about 13.6 grams to about 14.6 grams of beeswax or beeswax/DMSO mixture are used; and the beeswax/DMSO mixture if used contains from about 6.80 grams to about 13.432 grams of beeswax and from about 1.088 grams to about 7.3 grams of DMSO;
In step d), from about 5.64 grams to about 6.64 grams of glycerin is used; and
In step e), from about 0.000500 grams to about 0.200 grams of the API is used.

25. A transdermal composition prepared by the process of claim 1.
